(19) United States
(12) Patent Application Publication
(10) Pub. No.: US 2006/0140878 A1
(43) Pub. Date: Jun. 29, 2006

(54) Classified silica for improved cleaning and abrasion in dentifrices

(76) Inventors: John Mark Cornelius, Forest Hill, MD (US); Patrick Donald McGill, Darlington, MD (US)

Correspondence Address: William S. Parks, Esq.;J. M. Huber Corporation, 333 Thomall Street, Edison, NJ, 08837, US

(21) Appl. No.: 11/023,133

(22) Filed: Dec. 23, 2004

(51) Int. Cl. A61K 8/25

(52) U.S. Cl. 424/49

(57) ABSTRACT

A method of making precipitated silica abrasive compositions having excellent cleaning performance and lower abrasiveness with post-reactor sizing of the abrasive particles being performed via air classification techniques is provided. By targeting a specific particle size range, it has been determined that higher pellicle film cleaning levels may be achieved without also increasing the dentin abrasion properties of the silica products themselves. As a result, dentifrices including such classified abrasive silica products, and exhibiting particularly desirable cleaning benefits, can be provided for improved tooth polishing, whitening, and the like, without deleteriously affecting the hard tooth surfaces. Also encompassed within this invention also are products of this selective process scheme and dentifrices containing such classified silica products.

### FIELD OF THE INVENTION

This invention relates to a method of making abrasive compositions, and more particularly, it relates to a method of making precipitated silica abrasive compositions having excellent cleaning performance and lower abrasiveness with post-reactor sizing of the abrasive particles being performed via air classification techniques. By targeting a specific particle size range, it has been determined that higher pellicle film cleaning levels may be achieved without also increasing the dentin abrasion properties of the silica products themselves. As a result, dentifrices including such classified abrasive silica products, exhibiting particularly desirable cleaning benefits, can be provided for improved tooth polishing, whitening, and the like, without deleteriously affecting the hard tooth surfaces. Also encompassed within this invention also are products of this selective process scheme and dentifrices containing such classified silica products.

### BACKGROUND OF THE INVENTION

Toothpaste manufacturers strive to produce dentifrices with high cleaning and low abrasivity. Such formulators achieve this goal by incorporating abrasive substances into the toothpaste formulation. An abrasive substance has been included in conventional dentifrice compositions in order to remove various deposits, including pellicle film, from the surface of teeth. Pellicle film is tightly adherent and often contains brown or yellow pigments, which impart an unsightly appearance to the teeth. While cleaning is important, the abrasive should not be so aggressive so as to damage the teeth. Ideally, an effective dentifrice abrasive material maximizes pellicle film removal while causing minimal abrasion and damage to the hard tooth surfaces. Consequently, among other things, the performance of the dentifrice is highly sensitive to the abrasive polishing agent ingredient.

A number of water insoluble, abrasive polishing agents have been used or described for dentifrice compositions. These abrasive polishing agents include natural and synthetic abrasive particulate materials. The generally known synthetic abrasive polishing agents include amorphous precipitated silicas, silica gels, dicalcium phosphate and its dihydrate forms, calcium pyrophosphate and precipitated calcium carbonate (PCC). Other abrasive polishing agents for dentifrices have included chalk, magnesium carbonate, zirconium silicate, potassium metaphosphate, magnesium orthophosphate, tricalcium phosphate, and the like.

Synthetically produced amorphous precipitated silicas, in particular, have been used as abrasive components in dentifrice formulations due to their cleaning ability, relative safety, and compatibility with typical dentifrice ingredients, such as humectants, thickening agents, flavoring agents, anti-caries agents, and so forth. Synthetic precipitated silicas generally are produced by the de-stabilization and precipitation of amorphous silica from soluble alkaline silicate by the addition of a mineral acid and/or acid gases under conditions in which primary particles initially formed tend to associate with each other to form a plurality of aggregates (i.e., discrete clusters of primary particles), but without agglomeration into a three-dimensional gel structure. The resulting precipitate is separated from the aqueous fraction of the reaction mixture by filtering, washing, and drying procedures, and then the dried product is mechanically comminuted in order to provide a suitable particle size.

Such previously produced and utilized precipitated silica abrasives have been produced and provided for dentifrices generally in terms of overall cleaning and abrasive qualities. Although such previous products have accorded excellent benefits in these areas, it has been noted that certain limitations in terms of targeting certain lower abrasive levels without sacrificing pellicle film cleaning ability have existed as well, particularly as it concerns users susceptible to unwanted dentin abrasion at the gum line, as well as potential supplemental abrasive/cleaning silica products for more effective polishing and/or tooth whitening applications. As a result, there are areas within the dental silica materials industry in which improvements to such ends are desired.

Given the foregoing, there is a continuing need for a precipitated silica composition that provides excellent cleaning performance, but with lower abrasivity values, that can be included in a toothpaste composition. To that end, the following invention has proven to accord such coveted results.

### BRIEF SUMMARY OF THE INVENTION

The invention includes an amorphous precipitated silica composition, the silica composition having a median particle size of about 5 to about 15 microns, preferably from about 6 to about 10, and more preferably from about 7 to about 9, a particle size span of less than 2, preferably from about 1.25 to about 1.75, and more preferably from about 1.25 to about 1.40, and a particle size beta value greater than about 0.30, preferably from about 0.35 to about 0.50, and more preferably from about 0.40 to about 0.50.

The invention also includes a dentifrice comprising about 5 wt % to about 35 wt % of the amorphous precipitated silica composition noted above, and exhibiting an radioactive dentin abrasion (RDA) level between about 130 and 200 (preferably from about 130 to about 195), a pellicle film cleaning ratio (PCR) of between about 100 and 140 (preferably from about 110 to about 140), and a PCR:RDA ratio of from about 0.65 to about 1.1, preferably from about 0.68 to about 1.0.

Basically, it has been realized that providing low-structure abrasive silica materials within a concentrated range of specific particle sizes permits greater uniformity in performance during tooth cleaning with a dentifrice containing such materials. Likewise, providing such materials within the specific range of particle sizes permits targeting particular areas of tooth surfaces for proper cleaning without simultaneously exhibiting excessive abrasive levels.

### DETAILED DESCRIPTION OF THE INVENTION

All parts, percentages and ratios used herein are expressed by weight unless otherwise specified. All documents cited herein are incorporated by reference. The following describes preferred embodiments of the present invention, which provides silica for use in dentifrices, such as toothpastes. While the optimal use for this silica is in dentifrices, this silica may also be used in a variety of other consumer products.

By “mixture” it is meant any combination of two or more substances, in the form of, for example without intending to be limiting, a heterogeneous mixture, a suspension, a solution, a sol, a gel, a dispersion, or an emulsion.

By “dentifrices” it is meant oral care products such as, without intending to be limiting, toothpastes, tooth powders and denture creams.

By “particle size span” it is meant the cumulative diameter of the particles in the tenth volume percentile (D25) minus the cumulative volume at the ninetieth percentile (D90) divided by the diameter of the particles in the fiftieth volume percentile (D50), i.e. (D10−D90)/D50. A lower span value indicates a narrower particle size distribution.

By “particle size beta value” it is meant cumulative diameter of the particles in the twenty-fifth volume percentile (D25) divided by the diameter of the particles in the seventy-fifth volume percentile (D75), i.e. D25/D75. A higher beta value indicates a narrower particle size distribution.

The present invention relates to amorphous, precipitated silica compositions, also known as silicon dioxide, or SiO_{2,} which impart improved cleaning and abrasive characteristics when included within a toothpaste or dentifrice. These abrasive silicas not only clean teeth by removing debris and residual stains, but also function to polish tooth surfaces. Because the silicas of the present invention have been classified to remove fine particles which are believed to have less cleaning benefit and large particles which are believed to contribute to increased abrasion, they have a more narrow particle size distribution and are particularly useful for formulating a toothpaste that has excellent cleaning with lower abrasivity.

A sufficient amount of abrasive silica should be added to a toothpaste composition so that the radioactive dentin abrasion (“RDA') value of the toothpaste is between about 50 and about 250. At a RDA of less than 50, the cleaning benefits of the toothpaste will be minimal, while at a RDA of greater than 250, there is risk that the toothpaste will be so abrasive that it may damage the tooth dentin along the gum line. Preferably, the dentifrice should have a RDA value of at least about 50, such as between about 70 and 200.

The RDA of a toothpaste is dependent on the hardness of the abrasive, the abrasive particle size and the concentration of the abrasive in the toothpaste. The RDA is measured by the method described in the article “A Laboratory Method for Assessment of Dentifrice Abrasivity”, John J. Hefferren, in Journal of Dental Research, Vol. 55, no. 4 (1976), pp. 563-573. Silica abrasivity or hardness can also be measured by an Einlehner method, which is described in greater detail below.

By the present invention, abrasive amorphous silicas have been developed that not only have excellent cleaning performance but are also less abrasive. By using post reactor air classification equipment on spray dried and milled silica, an abrasive silica material may be produced that has relatively low RDA and Einlehner abrasion values over a given PCR range.

The silica compositions of the present invention are prepared according to the following process. In this process, an already formed dried silica is feed into an air classifier in order to separate the desired fraction from the finer and the coarser particles. The silica abrasive feed can be precipitated silica or silica gel of any structure, such as very low to medium structure, with very low to low structure precipitated silica preferred. Silica structure as used herein is described in the article “Cosmetic Properties and Structure of Fine-particle Synthetic Precipitated Silicas”, S. K. Wason, in the Journal of Soc. Cosmet. Chem., Vol. 29, (1978), pp. 497-521, which is incorporated herein by reference. Such inventive compositions include silica particles that exhibit a linseed oil absorption value of from about 50 ml/100 g to about 90 ml/100 g.

The silica feed can be produced according to the descriptions in U.S. Pat. Nos. 6,616,916, 5,869,028, 4,421,527, and 3,893,840, which are incorporated herein by reference.

The dried silica feed can be introduced into the classifier as an unmilled feedstock or milled before introduction to the classifier. The unmilled feedstock can be dried in any conventional equipment used for drying silica, e.g., spray drying, nozzle drying (e.g., tower or fountain), flash drying, rotary wheel drying or oven/fluid bed drying. The dried silica product generally should have a 1 to 15 wt % moisture level.

Alternately, the dried silica may be reduced in particle size with conventional grinding and milling equipment to obtain the desired particle size of between about 5 μm to about 25 μm, such as about 5 μm to about 15 μm, prior to introduction into the classifier. A hammer or pendulum mill may be used in one or multiple passes for comminuting and fine grinding can be performed by fluid energy or air-jet mill.

The dried silica is then subjected to air classification to yield the inventive silica with a narrow particle size distribution. Classification of the silica tightens the particle size distribution by removing the fine and large particles from the product. The classifier housing serves as a plenum into which the metered primary air is introduced through the inlet duct. This air enters the classifier rotor through the narrow gap between the tip of the two rotor halves and the stator. These opposing high velocity streams form a turbulent dispersing zone. Feed enters the system through the central tube, which is angled to the radial to minimize the distance of coarse particle injection into the vortex due to inertia The space between the outer edge of the blades and the periphery of the rotor forms the classification zone. Coarse product, which is rejected outward by the centrifugal field, is conveyed out of the classifier through the coarse outlet using a jet pump mounted on a cyclone. The cyclone overflow is returned to the classifier through the recycle port. Fine product leaves the classifier through the central outlet with the primary air flow. The silica is classified until the silica product has the desired particle size distribution.

Two criteria for describing the tightness of the particle size distribution are particle size span and beta values as measured using a Horiba laser light scattering instrument available from Horiba Instruments, Boothwyn, Pa. The size distribution of silica particles in a given composition may be represented on a Horiba which plots cumulative volume percent as a function of particle size. Where cumulative volume percent is the percent, by volume, of a distribution having a particle size of less than or equal to a given value and where particle size is the diameter of an equivalent spherical particle. The median particle size in a distribution is the size in microns of the silica particles at the 50% point on the Horiba for that distribution.

The width of the particle size distribution of a given composition can be characterized using a span ratio. The span ratio is defined as the cumulative diameter of the particles in the tenth volume percentile (D10) minus the cumulative volume at the ninetieth percentile (D90) divided by the diameter of the particles in the fiftieth volume percentile (D50), i.e. (D10−D90)/D50.

The particle size distribution is also characterized by a beta value. The particle size beta value is the cumulative diameter of the particles in the twenty-fifth volume percentile (D25) divided by the diameter of the particles in the seventy-fifth volume percentile (D75), i.e. D25/D75. A higher beta value indicates a narrower particle size distribution.

This abrasive, amorphous precipitated silica may then be incorporated into a dentifrice composition, e.g., toothpaste, either as the sole abrasive or with other abrasive components.

In addition to the abrasive component, the dentifrice may also contain several other ingredients commonly used in dentifrice making such as humectants, thickening agents, (also sometimes known as binders, gums, or stabilizing agents), antibacterial agents, fluorides, sweeteners, and co-surfactants.

Humectants serve to add body or “mouth texture” to a dentifrice as well as preventing the dentifrice from drying out. Suitable humectants include polyethylene glycol (at a variety of different molecular weights), propylene glycol, glycerin (glycerol), erythritol, xylitol, sorbitol, mannitol, lactitol, and hydrogenated starch hydrolyzates, as well as mixtures of these compounds.

Thickening agents are useful in the dentifrice compositions of the present invention to provide a gelatinous structure that stabilizes the toothpaste against phase separation. Suitable thickening agents include silica thickener, starch, glycerite of starch, gum karaya (sterculia gum), gum tragacanth, gum arabic, gum ghatti, gum acacia, xanthan gum, guar gum, veegum, carrageenan, sodium alginate, agar-agar, pectin, gelatin, cellulose, cellulose gum, carboxymethyl cellulose, hydroxyethyl cellulose, hydroxypropyl cellulose, hydroxymethyl, hydroxymethyl carboxypropyl cellulose, methyl cellulose, ethyl cellulose, sulfated cellulose, as well as mixtures of these compounds. Typical levels of binders are from about 0 wt % to about 15 wt % of a toothpaste composition.

Antibacterial agents may be included to reduce the presence of microorganisms to below known harmful levels. Suitable antibacterial agents include tetrasodium pyrophosphate, benzoic acid, sodium benzoate, potassium benzoate boric acid phenolic compounds such as betanapthol, chlorothymol, thymol, anethole, eucalyptol, carvacrol, menthol, phenol, amylphenol, hexylphenol, heptylphenol, octylphenol, hexylresorcinol, laurylpyridinium chloride, myristylpyridinium chloride, cetylpyridinium fluoride, cetylpyridinium chloride, cetylpyridinium bromide. If present, the level of antibacterial agent is preferably from about 0.1 wt % to about 5 wt % of the toothpaste composition.

Sweeteners may be added to the toothpaste composition to impart a pleasing taste to the product. Suitable sweeteners include saccharin (as sodium, potassium or calcium saccharin), cyclamate (as a sodium, potassium or calcium salt), acesulfame-K, thaumatin, neohisperidin dihydrochalcone, ammoniated glycyrrhizin, dextrose, levulose, sucrose, mannose, and glucose.

The toothpaste will also preferably contain fluoride salts to prevent the development and progression of dental caries. Suitable fluoride salts include sodium fluoride, potassium fluoride, zinc fluoride, stannous fluoride, zinc ammonium fluoride, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, and sodium monofluorophosphate. Typical levels of fluoride salts are from about 0.1 wt % to about 5 wt %.

Surfactants may also be included as additional cleansing and foaming agents, and may be selected from anionic surfactants, zwitterionic surfactants, nonionic surfactants, amphoteric surfactants, and cationic surfactants. Anionic surfactants are preferred, such as metal sulfate salts, such as sodium lauryl sulfate.

The dentifrices disclosed herein may also contain a variety of additional ingredients such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, whitening agents, colorants, flavorants, and preservatives.

Finally, water provides the balance of the composition in addition to the additives mentioned. The water is preferably deionized and free of impurities. The dentifrice will comprise from about 10 wt % to about 40 wt % of water, preferably from 20 to 35 wt %.

### PREFERRED EMBODIMENTS OF THE INVENTION

The invention will now be described in more detail with respect to the following, specific, non-limiting examples.

### COMPARATIVE EXAMPLES A-B

In order to show the improvement of the present invention, 2 commercial precipitated silicas ZEODENT® 103 and ZEODENT® 124, Comparative Example A and Comparative Example B, respectively, were characterized. These products are available form J. M. Huber Corporation, Edison, N.J. Physical properties of these examples are summarized below in Table 2.

### EXAMPLES 1-2

In Examples 1 and 2, silicas suitable for use in dentifrices as well as other products, were prepared according to the present invention.

The starting material for Example 1 silica was Comparative Example A, ZEODENT® 103. The dried precipitated silica was then air classified, under the conditions listed in Table I, with multiple passes through a High Efficiency Centrifugal Air Classifier (Model 250) manufactured by CCE Technologies, Inc., Cottage Grove, Minn.

The starting material for Example 2 was Comparative Example B, ZEODENT® 124 silica which was first milled. The milled precipitated silica was then air classified, under the conditions listed in Table I.

TABLE 1

| | Example 1 | Example 2 |
|---|---|---|
| Rotor Speed (rpm) | 1550 | 1550 |
| Flow Delta P (in. H₂O) | 4.5 | 4.5 |
| Air Flow (scfm) | 247 | 247 |
| Ejector Pressure (psig) | 50 | 50 |
| Class. Delta P (in. Hg) | 6.5 | 7.2 |

After being prepared as set forth above, several properties of the particulate silica, including median particle size, mean particle size, particle size beta value, particle size span, % 325 mesh residue, BET surface area, CTAB surface area, oil absorption, and Einlehner abrasion were then measured.

Particle size measurements were determined using a Model LA-910 laser light scattering instrument available from Horiba Instruments, Boothwyn, Pa. A laser beam is projected through a transparent cell which contains a stream of moving particles suspended in a liquid. Light rays which strike the particles are scattered through angles which are inversely proportional to their sizes. The photodetector array measures the quantity of light at several predetermined angles. Electrical signals proportional to the measured light flux values are then processed by a microcomputer system to form a multi-channel histogram of the particle size distribution. Median and mean particle sizes were measured in addition to the particle size span ((D10−D90)/D50) and beta values (D25/D75).

The %325 sieve residue was determined by weighing 50 g silica into a 1-liter beaker containing 500-600 ml water. The silica particles were allowed to settle into the water, then mixed well until all the material was dispersed. The water pressure was adjusted through the spray nozzle (Fulljet 9.5, 3/8 G, 316 stainless steel, Spraying Systems Company) to 20-25 psi. The sieve screen cloth (325 mesh screen, 8″ diameter) was held 4-6 inches below the nozzle and, while spraying, the contents of the beaker were gradually poured onto the 325 mesh screen. The remaining material from the walls of the beaker was rinsed and poured onto the screen. Washing occurred for 2 minutes, moving the spray from side to side in the screen using a sweeping motion. After spraying for 2 minutes (all particles smaller than the screen opening should have passed through the screen), the residue retained on the screen was washed to one side, and then transferred into a pre-weighed aluminum weighing dish by washing with water from a squirt bottle. The minimum amount of water needed was used to be sure all the residue is transferred into the weighing dish. The dish was allowed to stand 2-3 minutes (residue settles), then the clear water was decanted off the top. The dish was placed in an oven (“Easy-Bake” infrared oven or conventional oven set to 105° C.) and dried until the residue was dried to a constant weight. The dry residue sample and dish was re-weighed. Calculation of % 325 residue was done as follows:

$$\%325 residue = \frac{weight of residue , g}{sample weight , g} \times 100$$

The BET surface area was determined by the BET nitrogen adsorption methods of Brunaur et al., J. Am. Chem. Soc., 60, 309 (1938).

The CTAB external surface area of silica is determined by absorption of CTAB (cetyltrimethylammonium bromide) on the silica surface, the excess separated by centrifugation and determined by titration with sodium lauryl sulfate using a surfactant electrode. The external surface of the silica is determined from the quantity of CTAB adsorbed (analysis of CTAB before and after adsorption)

Specifically, about 0.5 g of silica is accurately weighed and placed in a 250-ml beaker with 100.00 ml CTAB solution (5.5 g/L), mixed on an electric stir plate for 30 minutes, then centrifuged for 15 minutes at 10,000 rpm. One ml of 10% Triton X-100 is added to 5 ml of the clear supernatant in a 100-ml beaker. The pH is adjusted to 3.0-3.5 with 0.1 N HCI and the specimen is titrated with 0.0100 M sodium lauryl sulfate using a surfactant electrode (Brinkmann SUR1501-DL) to determine the endpoint.

The oil absorption was measured using linseed oil by the rubout method. In this test, oil is mixed with a silica sample and rubbed with a spatula on a smooth surface until a stiff putty-like paste is formed. By measuring the quantity of oil required to have a paste mixture, which will curl when spread out, one can calculate the oil absorption value of the silica—the value which represents the volume of oil required per unit weight of silica to completely saturate the silica sorptive capacity. Calculation of the oil absorption value was done as follows:

$$\begin{matrix}Oil absorbtion = \frac{ml oil absorbed}{weight of silica , g} \times 100 \\ = ml oil / 100 g silica\end{matrix} \qquad ( II )$$

The Brass Einlehner (BE) Abrasion value was measured through the use of an Einlehner AT-1000 Abrader. In this test, a Fourdrinier brass wire screen is weighed and exposed to the action of a 10% aqueous silica suspension for a fixed number of revolutions, and the amount of abrasion is then determined as milligrams brass lost from the Fourdrinier wire screen per 100,000 revolutions. Disposable supplies required for this test (brass screens, wear plates and PVC tubing) are available from Duncan Associates, Rutland, Vt. and sold as an “Einlehner Test Kit”. Specifically, brass screens (Phosphos Bronze P. M.) were prepared by washing in hot, soapy water (0.5% Alconox) in an ultrasonic bath for 5 minutes, then rinsed in tap water and rinsed again in a beaker containing 150 ml water set in an ultrasonic bath. The screen is rinsed again in tap water, dried in an oven set at 105° C. for 20 minutes, cooled in a desiccator and weighed. Screens were handled with tweezers to prevent skin oils from contaminating the screens. The Einlehner test cylinder is assembled with a wear plate and weighed screen (red line side down—not abraded side) and clamped in place. The wear plate is used for about 25 tests or until worn badly; the weighed screen is used only once.

A 10% silica slurry, prepared by mixing 100 g silica with 900 g deionized water, was poured into the Einlehner test cylinder. Einlehner PVC tubing was placed onto the agitating shaft. The PVC tubing has 5 numbered positions. For each test, the position of the PVC tubing is incremented until it has been used five times, then discarded. The Einlehner abrasion instrument is re-assembled and the instrument set to run for 87,000 revolutions. Each test takes about 49 minutes. After the cycle is completed, the screen is removed rinsed in tap water, placed in a beaker containing water and set in an ultrasonic bath for 2 minutes, rinsed with deionized water and dried in an oven set at 105° C. for 20 minutes. The dried screen is cooled in a desiccator and reweighed. Two tests are run for each sample and the results are averaged and expressed in mg lost per 100,000 revolutions. The result, measured in units of mg lost per 100,000 revolutions, for a 10% slurry can be characterized as the 10% brass Einlehner (BE) abrasion value. The results of these measurements and tests are summarized below in Table 2.

TABLE 2

| | | | Comparative | Comparative |
|---|---|---|---|---|
| | Example 1 | Example 2 | Example A | Example B |
| Median Particle | 8.1 | 8.0 | 9.4 | 8.3 |
| Size (μm) | | | | |
| Mean Particle | 8.2 | 8.7 | 13.1 | 11.2 |
| Size (μm) | | | | |
| Particle Size | 0.45 | 0.41 | 0.19 | 0.24 |
| Beta Value | | | | |
| Particle Size | 1.47 | 1.61 | 3.01 | 2.73 |
| Span | | | | |
| % 325 Residue | 0.0 | 0.0 | 1.2 | 0.4 |
| BET Surface | — | 53 | 39 | 73 |
| Area (m²/g) | | | | |
| CTAB Surface | 22 | 48 | 32 | 42 |
| Area (m²/g) | | | | |
| Oil Absorption | 59 | 74 | 70 | 72 |
| (ml/100 g) | | | | |
| Einlehner | 13.10 | 6.38 | 18.92 | 8.57 |
| Abrasion (mg) | | | | |

As can be seen in Table 2, the silicas prepared in Examples 1-2 have smaller median and mean particle sizes as compared to Comparative Examples A-B. Examples 1-2 silicas have narrower particles size distributions as indicated by their lower particle size spans and higher particle size beta values. Examples 1-2 also have lower Einlehner abrasion values while still being sufficiently abrasive to produce toothpaste with acceptable or good cleaning performance. By contrast, Comparative Examples A-B exhibit broader particle size distributions and are more abrasive.

To demonstrate their efficacy in consumer products, the silica abrasives of Examples 1-2 were incorporated as powders into four different toothpaste compositions (numbers 1-4), each at a 20% and 35% silica loading level. The performance of these compositions was then compared with the performance of toothpaste compositions 5-8 formulated with Comparative Example A-B silicas each at 20% and 35% silica loading levels. The eight toothpaste compositions are set forth in Table 3, below.

These toothpaste samples were prepared as follows. A first mixture was formed by combining the following components: glycerin and sorbitol, polyethylene glycol (CARBOWAX® 600, from the Union Carbide Corporation, Danbury, Conn.), carboxymethylcellulose (such as CEKOL® 2000 from Noviant, Arnhem, The Netherlands, or CMC-7MXF from the Aqualon division of Hercules Corporation, Wilmington, Del.), and then stirring the first mixture until the components dissolved. A second mixture was formed by combining the following components: deionized water, tetrasodium pyrophospate, sodium saccharin, sodium fluoride, and then stirring until the components are dissolved. The first and second mixtures were then combined while stirring to form a premix. The premix was placed into a Ross mixer (model 130LDM, Charles Ross & Co., Haupeauge, N.Y.), silica thickener, titanium dioxide, and silica abrasive added to the premix, and the premix mixed without vacuum. Then 30 inches of vacuum was drawn and each sample mixed for 15 minutes, and then sodium lauryl sulfate and flavor was added. The resulting mixture was stirred for 5 minutes at a reduced mixing speed. The eight different toothpaste compositions were prepared according to the following formulations, wherein the amounts are gram units:

TABLE 3

| | Toothpaste Composition Number | | | | | | | |
|---|---|---|---|---|---|---|---|---|
| Ingredients | 1 | 2 | 3 | 4 | 5 | 6 | 7 | 8 |
| Glycerin, 99.5% | 11 | 9.683 | 11 | 9.683 | 11 | 9.683 | 11 | 9.683 |
| Sorbitol, 70% | 40.007 | 28.718 | 40.007 | 28.718 | 40.007 | 28.718 | 40.007 | 28.718 |
| Deionized Water | 20 | 17.806 | 20 | 17.806 | 20 | 17.806 | 20 | 17.806 |
| Carbowax 600 | 3 | 3 | 3 | 3 | 3 | 3 | 3 | 3 |
| CMC-7MXF | 1.2 | 1 | 1.2 | 1 | 1.2 | 1 | 1.2 | 1 |
| Tetrasodium | 0.5 | 0.5 | 0.5 | 0.5 | 0.5 | 0.5 | 0.5 | 0.5 |
| Pyrophosphate | | | | | | | | |
| Sodium Saccharin | 0.2 | 0.2 | 0.2 | 0.2 | 0.2 | 0.2 | 0.2 | 0.2 |
| Sodium Fluoride | 0.243 | 0.243 | 0.243 | 0.243 | 0.243 | 0.243 | 0.243 | 0.243 |
| Zeodent ®165 silica | 1.5 | 1.5 | 1.5 | 1.5 | 1.5 | 1.5 | 1.5 | 1.5 |
| thickener | | | | | | | | |
| Example 1 abrasive | 20 | 35 | — | — | — | — | — | — |
| Example 2 abrasive | — | — | 20 | 35 | — | — | — | — |
| Comparative | — | — | — | — | 20 | 35 | — | — |
| Example A abrasive | | | | | | | | |
| Comparative | — | — | — | — | — | — | 20 | 35 |
| Example B abrasive | | | | | | | | |
| TiO₂ | 0.5 | 0.5 | 0.5 | 0.5 | 0.5 | 0.5 | 0.5 | 0.5 |
| Sodium Lauryl | 1.2 | 1.2 | 1.2 | 1.2 | 1.2 | 1.2 | 1.2 | 1.2 |
| Sulfate | | | | | | | | |
| Flavor | 0.65 | 0.65 | 0.65 | 0.65 | 0.65 | 0.65 | 0.65 | 0.65 |
| Total | 100.00 | 100.00 | 100.00 | 100.00 | 100.00 | 100.00 | 100.00 | 100.00 |

After toothpaste compositions 1-8 were prepared, as above, RDA and PCR properties were determined as follows. The Radioactive Dentin Abrasion (RDA) values of the precipitated silica compositions used in this invention are determined according to the method set forth by Hefferen, Journal of Dental Res., July-August 1976, 55 (4), pp. 563-573, and described in Wason U.S. Pat. Nos. 4,340,583, 4,420,312 and 4,421,527, which publications and patents are incorporated herein by reference.

The PCR test used to analyze the toothpaste compositions is described in “In Vitro Removal of stain With Dentifrice” G. K. Stookey, et al., J. Dental Res., 61, 1236-9, 1982.

PCR and RDA were measured 3 times for each of the toothpaste compositions and the results averaged. The average results of the RDA and PCR measurements, as well as the ratios of such measurements, are summarized in Table 4, below.

TABLE 4
Toothpaste Properties

| | Toothpaste Composition No. | | | | | | | |
|---|---|---|---|---|---|---|---|---|
| | 1 | 2 | 3 | 4 | 5 | 6 | 7 | 8 |
| PCR | 123 | 128 | 111 | 116 | 115 | 123 | 109 | 122 |
| RDA | 181 | 195 | 144 | 144 | 225 | 249 | 170 | 205 |
| PCR/RDA | 0.68 | 0.66 | 0.77 | 0.81 | 0.51 | 0.49 | 0.64 | 0.60 |

It is seen in Table 4, that the toothpastes containing the inventive silicas (Toothpaste Compositions 1-4) in all cases had equivalent PCR values as compared to the corresponding control toothpastes (Toothpaste Compositions 5-8). Surprisingly, the RDA values for the inventive Toothpaste Compositions 1-4 were 26 to 61 points lower than the corresponding control Toothpaste Compositions 5-8. Furthermore, the ratios were calculated to be significantly higher for the inventive classified silica products than for the comparative silica products showing a marked improvement over the currently practiced abrasives.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A composition comprising amorphous precipitated silica particles, wherein said silica particles present within said composition exhibit a median particle size of about 5 to about 15 microns, a particle size span of less than 2, and a particle size beta value of greater than 0.3.

2. The composition of claim 1 wherein said silica particles present within said composition exhibit a median particle size of about 6 to about 10 microns, a particle size span of from about 1.25 to about 1.75, and a particle size beta value of from about 0.35 to about 0.50.

3. The composition of claim 2 wherein said silica particles present within said composition exhibit a median particle size of about 7 to about 9 microns, a particle size span of from about 1.25 to about 1.65, and a particle size beta value of from about 0.40 to about 0.50.

4. The composition of claim 1, wherein said silica particles exhibit a linseed oil absorption value of from about 50 ml/100 g to about 90 ml/100 g.

5. The composition of claim 2, wherein said silica particles exhibit a linseed oil absorption value of from about 50 ml/100 g to about 90 ml/100 g.

6. The composition of claim 3, wherein said silica particles exhibit a linseed oil absorption value of from about 50 ml/100 g to about 90 ml/100 g.

7. A dentifrice formulation comprising about 5 wt % to about 35 wt % of the composition of claim 1.

8. A dentifrice comprising about 5 wt % to about 35 wt % of the composition of claim 2.

9. A dentifrice comprising about 5 wt % to about 35 wt % of the composition of claim 3.

10. A dentifrice comprising about 5 wt % to about 35 wt % of the composition of claim 4.

11. A dentifrice comprising about 5 wt % to about 35 wt % of the composition of claim 5.

12. A dentifrice comprising about 5 wt % to about 35 wt % of the composition of claim 6.

13. A dentifrice comprising the composition of claim 1, wherein said dentifrice exhibits a radioactive dentin abrasion (RDA) level between about 130 and 200 and a pellicle film cleaning ratio (PCR) of between about 100 and 140.

14. The dentifrice of claim 13, wherein said dentifrice exhibits a RDA level between about 130 to about 195 and a PCR of between about 110 and about 140.

15. A dentifrice comprising the composition of claim 2, wherein said dentifrice exhibits a RDA level between about 130 and 200 and a PCR of between about 100 and 140.

16. The dentifrice of claim 15, wherein said dentifrice exhibits a RDA level between about 130 to about 195 and a PCR of between about 110 and about 140.

17. A dentifrice comprising the composition of claim 3, wherein said dentifrice exhibits a RDA level between about 130 and 200 and a PCR of between about 100 and 140.

18. The dentifrice of claim 17, wherein said dentifrice exhibits a RDA level between about 130 to about 195 and a PCR of between about 110 and about 140.

19. The dentifrice of claim 13, wherein said dentifrice exhibits a PCR/RDA of from about 0.65 to about 1.1.

20. The dentifrice of claim 13, wherein said dentifrice exhibits a PCR/RDA of from about 0.68 to about 1.0.
